(19) United States
(12) Patent Application Publication
(10) Pub. No.: US 2016/0107967 A1
(43) Pub. Date: Apr. 21, 2016

(54) METHOD FOR THE DEPOLYMERIZATION OF LIGNIN

(71) Applicant: STICHTING DIENST LANDBOUWKUNDIG ONDERZOEK, Wageningen (NL)

(72) Inventors: Daniël Stephan VAN ES, Bennekom (NL); Frits VAN DER KLIS, Sliedrecht (NL); Jacobus VAN HAVEREN, Ede (NL); Richard Johannes Antonius GOSSELINK, Zelhem (NL)

(73) Assignee: Stichting Dienst Landbouwkundig Onderzoek, Wageningen (NL)

(21) Appl. No.: 14/782,947

(22) PCT Filed: Apr. 7, 2014
(86) PCT No.: PCT/NL2014/050216

§ 371 Date: Oct. 7, 2015

(30) Foreign Application Priority Data
Apr. 8, 2013 (EP) 13162749.9

(51) Int. Cl. C07C 37/50; C07C 41/18

(52) U.S. Cl. CPC C07C 37/50; C07C 41/18

(57) ABSTRACT

A catalytic method is disclosed for the valorization of lignin. The method comprises subjecting lignin to a catalyzed hydrothermal conversion reaction, said reaction being conducted in the presence of water at an alkaline pH>8 and a temperature of 200° C.-300° C., under the influence of a noble metal catalyst comprising a carbon support. The same reaction can also be applied to subsequently defunctionalize one or more phenolic compounds, notably guaiacol, so as to produce six-membered cyclic hydrocarbons, such as phenol.

### FIELD OF THE INVENTION

The invention is in the field of the catalytic valorization of lignin. Particularly, the invention pertains to a process for the hydrothermal conversion of lignin and the in-situ catalytic upgrading of depolymerized lignin.

### BACKGROUND OF THE INVENTION

Biomass is recognized as a source for the renewable production of fuels, chemicals, and energy. In view hereof, and taking into account the limits to fossil fuel sources, the demand for bio-refineries is on the rise. However, in order to provide a greater economic viability of these refineries, new processes need to be developed for the production of high value chemicals and fuel (additives) from biomass.

In the art, attention is increasingly drawn to lignin as a potential source for valuable chemicals. Next to cellulose and hemicellulose, lignin is one of the three major components of lignocellulosic biomass. Lignin is a known source for valuable aromatic, and particularly phenolic, compounds. A challenge, however, is to actually produce a limited set of aromatic compounds, and other compounds such as cyclohexanone, from lignin in a sufficient specificity, and on the basis of a desirable conversion. The application of phenol(derivatives) in the production of polymers requires high purities of the building blocks. The existing technology for producing phenols and aromatics from lignin biomass, yields complex mixtures which are either unsuitable for polymer chemistry or require unrealistic downstream processing costs. So it is desirable to obtain a limited number of compounds that can be separated and/or purified by conventional industrial separation/purification techniques such as distillation.

It is known to depolymerize lignin by methods involving the use of a catalyst and the addition of external hydrogen or hydrogen donors. The latter is undesirable, particularly considering that the main idea behind lignin valorization resides in the economics of using renewable sources such as biomass. The need to externally supply (non-renewable) hydrogen is thus at odds with the basic philosophy of biomass valorization.

A reference dealing with this issue is US 2010/0137665. Herein a process is presented for the production of high value chemicals from lignin. The process comprises combining several internal steps to use the hydrogen generated by the process, rather than adding an external source of hydrogen. In the process, lignin is combined with water and a catalyst to form an intermediate stream comprising deoxygenated lignin and light oxygenates. The latter are decomposed to form a second intermediate stream comprising hydrogen. Said second intermediate stream is contacted with a hydrogenation catalyst to form a product stream. A drawback of this process is the complex three-stage nature thereof. Moreover, the process does not address the key desires of improving conversion rate as well as product specificity.

Another reference on the production of aromatic compounds from lignin is Zakzeski and Weckhuysen, ChemSusChem 2011, 4, 369-378. Herein the aqueous phase reforming of lignin is discussed, which is a process wherein lignin is solubilized in water at about 200° C. and subjected to a Pt/Al₂O₃ catalyst in the presence of sulfuric acid. The method is shown to result in a wide variety of phenolic compounds, in modest yields.

Another reference wherein hydrogen is applied, as a 40 bar atmosphere, is NING YAN ET AL: “Selective Degradation of Wood lignin over Noble-Metal Catalysts in a Two-Step Process”, CHEMSUSCHEM, vol. 1, no. 7, 21 Jul. 2008, pages 626-629, XP055076054 (“Yan et al.”). Yan et al. describes a 2-step process for the production of C8-C18 alkanes and methanol from lignin. The final products obtained are alkanes, rather than aromatic alcohols as desirably obtained in accordance with the invention. It should be noted that, during the process of obtaining alkanes, Yan et al. pass along intermediates which are alkylated phenols. These are not the desired products of the invention.

In order to improve the valorization from lignin, it would be desired to obtain a more narrow product distribution, and a better yield than available in the art. Also, it would be desired to provide a process that does not require the addition of hydrogen. Particularly it is desired to provide a process that allows obtaining directly useful, preferably non-alkylated phenolic compounds, such as phenol, 2-methoxy phenol, 2-6-dimethoxy phenol, veratrole (1,2-dimethoxy benzene), or 4-hydroxy, 3,5-dimethoxyacetophenone (acetosyringone).

### SUMMARY OF THE INVENTION

In order to address one or more of the foregoing desires the invention, in one aspect, provides a method for the depolymerization of lignin, wherein phenolic compounds are obtained by a method comprising subjecting lignin to a catalyzed hydrothermal conversion reaction, said reaction being conducted in the presence of water at an alkaline pH>8 and a temperature of 200° C.-300° C., under the influence of a noble metal catalyst on a carbon support.

In another aspect, the invention presents a process for the production of phenolic compounds from lignin, wherein the process comprises subjecting lignin to a depolymerization method as described above, and isolating one or more phenolic compounds (including alkoxy derivatives), particularly selected from the group consisting of phenol, 2-methoxy phenol, 2-6-dimethoxy phenol, veratrole (1,2-dimethoxy benzene), and 4-hydroxy, 3,5-dimethoxyacetophenone (acetosyringone).

In yet another aspect, the invention provides a process for the production of six-membered cyclic hydrocarbon compounds from lignin, wherein the process comprises subjecting lignin to a depolymerization method as described above, so as to obtain one or more phenolic compounds, and subjecting the phenolic compounds to a catalyzed hydrothermal defunctionalization reaction, said reaction being conducted in the presence of water at an alkaline pH>8 and a temperature of 200° C.-300° C., under the influence of a noble metal catalyst on a carbon support.

In a still further aspect, the invention provides a process for the defunctionalization of one or more phenolic compounds selected from the group consisting of phenol, C₁₋₆ alkyl alkoxy phenols, and acetosyringone (4-hydroxy-3,5-dimethoxyacetophenone), comprising subjecting the one or more phenolic compounds to a catalyzed hydrothermal defunctionalization reaction, said reaction being conducted in the presence of water at an alkaline pH>8 and a temperature of 200° C.-300° C., under the influence of a noble metal catalyst on a carbon support.

### DETAILED DESCRIPTION OF THE INVENTION

The invention, in a broad sense, is based on the recognition that the judicious choice for an alkaline pH, in combination with a noble metal catalyst on a carbon support, brings about unexpected advantages in the depolymerization and defunctionalization of lignin.

Without wishing to be bound by theory, the present inventors believe that the alkaline pH is instrumental in obtaining the desired product composition. E.g., the aforementioned process of Yan et al, which results in a different product composition, expressly teaches the addition of an acid (H₃PO₄) as this would improve the efficiency of the reaction. Yan et al., however, does not result in the products preferred according to the present invention. Generally, the process of the invention will not be conducted under a hydrogen atmosphere. In some embodiments, the process of the invention is conducted under an oxygen-containing atmosphere such as air, or under an inert atmosphere, such as nitrogen.

These alkaline circumstances refer to a pH above 8. Preferably the pH is in a range of from 9 to 12. More preferably, a pH of from 10 to 11 is applied. The pH is set in a conventional manner. The system subjected to the depolymerization and/or defunctionalization reaction is aqueous, particularly in the form of an aqueous suspension or solution. The pH is raised by adding a suitable base, such as sodium hydroxide or potassium hydroxide, until the desired pH is reached. Suitable bases include alkaline and alkaline-earth hydroxides or oxides, and ammonia. A preferred base is aqueous sodium hydroxide.

The reaction is catalyzed by a supported noble metal catalyst. In order to enable the use of high temperature aqueous alkaline conditions, the support is judiciously chosen so as to be capable of withstanding the reaction conditions. To this end, carbon is chosen. Carbon is available in several forms as a catalyst support, e.g. as active carbon, particularly as a slurry of activated carbon particles, or, e.g., in the form of carbon nanotubes (CNT), which can be multi walled or single walled CNF, carbon nano-fibers (CNF), graphite, or graphene. Reference is further made to Serp and Figueiredo (Editors), Carbon Materials for Catalysis, Wiley (2009) and to Rodriguez-Reinoso, Carbon Vol. 36, pp. 159-175, 1998.

The active part of the catalyst is a noble metal. Noble metals are ruthenium, rhodium, palladium, indium, platinum, and gold. Preferred metal catalysts for use in the present invention are ruthenium, rhodium, palladium, platinum, iridium, and gold. The most preferred catalytic metal is palladium. The catalyst system can be monometallic in nature, but also bimetallic, e.g. AuPd on carbon. As is customary in the art, promoters can be present in addition to the noble metals.

The depolymerization and defunctionalization reactions can be conducted in equipment, and according to methods, well known to the skilled person. Typically, lignin is obtained from biomass and is provided in such a way that it can be easily suspended in water. Exactly in which form the lignin is provided, will generally depend on the source of lignin. Suitable lignins include Kraft, soda- and organosolv lignins. The lignin used can also be fractionated lignin. Methods for fractionation of lignin are known to the skilled person. Suitable methods include ultrafiltration and selective extraction or precipitation. References to lignin fractionation include: Gosselink et al., Holzforschung 64, pp. 193-200, 2010, and Toledano et al., Chemical Engineering Journal 157 (2010) 93-99.

The equipment will generally be a reactor provided with means to heat and cool as desired. In view of the presence of liquid water in the reaction system, preferred reactors are batch reactors, particularly batch pressure reactors, plug-flow reactors (such as fixed bed reactors, continuous reactors, or tube reactors), and continuous stirred tank reactors (CSTR). Such suitable reactors, and the manners in which to introduce supported catalyst systems therein, are known to the skilled person.

It has, surprisingly, been found that already at very short contact times a desired conversion of lignin is obtained Thus, when the lignin is subjected to the influence of the catalyst, at a temperature of 200° C.-300° C., a “zero” contact time already brings about a depolymerization, respectively defunctionalization reaction. Although thus the reaction is instantaneous, it is preferred to ensure a longer reaction time so as to improve conversion and yield. The reaction is preferably conducted for a period of from 1 hour to 6 hours, more preferably of from 2 hours to 6 hours. The reaction is preferably conducted at a temperature of from 225° C. to 275° C., and more preferably a temperature of 250° C. is chosen.

As mentioned above, the present invention initially pertains to a method for the depolymerization of lignin. Lignin comprises a complex polymeric structure, generally without a recognizable primary structure, comprising a plurality of phenyl rings cross-linked through various linkages. In fact, lignin contains up to fourteen different types of linkages, mainly ether bonds, that cleave preferably under hydrothermal or supercritical water conditions (see the paper by Zakzeski and Weckhuysen referred to above). During hydrothermal decomposition, reactive species like aldehydes are believed to be formed, resulting in re-condensation of the monomeric/oligomeric phenolic species to highly condensed insoluble matter (char). The depolymerization in fact amounts to a breaking down of the lignin structure into phenolic compounds, i.e. the monocyclic aromatic residues of the lignin structure. Due to the lignin structure, these phenolic compounds will generally comprise functional groups in addition to the phenolic hydroxyl, e.g. of the ether, carbonyl, and carboxyl type. The number of variants of phenolic compounds possibly obtained from lignin depolymerization is huge, i.e., tens to hundreds of such compounds can be envisaged. In obtaining valuable chemicals through this route, it is therefore not only of importance to break-down the lignin structure (depolymerization) but also to bring about defunctionalization (so as to end up with a valuable bulk chemical such as phenol, rather than with a complex mixture of functionalized phenolic compounds).

In depolymerizing and defunctionalizing lignin, one generally has to balance a desired conversion against a desired product specificity. For, methods that bring about a relatively high degree of conversion, are prone to result in an undesirably high number of different, mostly monocyclic, aromatic compounds, or even in tar-like structures (char) as mentioned above. On the other hand, methods that seek to produce only, e.g., up to five phenolic compounds, are almost inevitably based on reaction of a low conversion.

The method of the invention surprisingly brings about a desirable degree of conversion, combined with a desirable product specificity. The phenolic compounds obtained at a level of a few percentages to tens of percentages are mostly limited to phenol, 2-methoxy phenol, 2-6-dimethoxy phenol, veratrole (1,2-dimethoxy benzene), and 4-hydroxy-3,5-dimethoxyacetophenone (acetosyringone). Moreover, the most desired phenolic compounds, viz. phenol and 2-methoxy phenol (guaiacol) are also the most abundant phenolic compounds obtained. For completeness sake: where in this description it is spoken of “phenolic” compounds, this includes compounds that have one or more alkoxy groups rather than a hydroxyl group. In the compounds subjected to processes of the invention, alkoxy is preferably C₁₋₃ alkoxy, and more preferably methoxy.

As a further benefit of the method of the invention, it is also capable of being used for the defunctionalization of the limited number of phenolic compounds obtained. This aspect of the invention is applicable to such phenolic compounds also if obtained from other biomass resources such as e.g. tannins, aromatic amino acids, to phenolics derived from sugars or fatty acids or to phenolics from non-renewable resources. Preferably, the phenolic compounds are obtained from lignin, most preferably resulting from the above-described process of the invention.

The phenolic compounds are selected from the group consisting of phenol, C₁₋₆ alkyl alkoxy phenols, preferably C₁₋₆ alkyl methoxy phenols, and acetosyringone (4-hydroxy-3,5-dimethoxyacetophenone). In the C₁₋₆ alkyl alkoxy phenols, the alkyl and the alkoxy, each independently, preferably refer to C₁₋₃ alkyl or C₁₋₃ alkoxy, and more preferably to methyl or methoxy. Preferred alkyl alkoxy phenols are selected from the group consisting of guaiacol (2-methoxy phenol), 2-6-dimethoxy phenol, 4 alkyl phenols, 4 alkyl 2-methoxy phenols, and 4 alkyl 2,6-dimethoxy phenols, with alkyl being C₁₋₆ alkyl, and preferably C₁₋₃ alkyl.

Preferably, according to the invention, the aforementioned defunctionalization will be conducted in addition to depolymerization. The latter particularly serves to enhance the economic potential for lignin as a source of desired bulk chemicals, viz. preferably phenol or mixtures of phenolic compounds for fuel additive applications.

In one aspect, the invention provides a process for the production of phenolic compounds from lignin, the process comprising subjecting lignin to a depolymerization method as described above, and isolating one or more phenolic compounds. Typical phenolic compounds so obtained are phenol, guaiacol (2-methoxy phenol, 2-6-dimethoxy phenol, veratrole (1,2-climethoxy benzene, and 4-hydroxy, 3,5-dimethoxyacetophenone (acetosyringone). Desirably, at least 2-methoxy phenol is isolated for further chemical processing into phenol.

In connection herewith, the invention provides a process for the production of phenol from lignin, the method comprising subjecting lignin to a depolymerization method as described above, so as to obtain one or more phenolic compounds comprising 2-methoxy phenol, and subjecting 2-methoxy phenol to a catalyzed hydrothermal defunctionalization reaction, said reaction being conducted in the presence of water at an alkaline pH>8 and a temperature of 200° C.-300° C., under the influence of a noble metal catalyst comprising a carbon support.

The foregoing method can also be conducted using one or more other phenolic compounds obtained from lignin depolymerization, such as syringol (2,5-dimethoxy phenol).

It is also conceivable, particularly by virtue of the high product specificity of the lignin depolymerization reaction of the invention, to refrain from isolating one or more phenolic compounds. In that case, the mixture resulting from the aforementioned depolymerization reaction, will be subjected to defunctionalization. In this embodiment, the two reaction processes (depolymerization and defunctionalization) can be conducted in two separate reactors. However, it is also conceivable to subject lignin to a two-stage process in one go, by first conducting the depolymerization and then conducting the defunctionalization in the same reactor.

The latter effectively results in an in situ method of valorizing lignin to the extent that six-membered cyclic hydrocarbons are produced from it. These hydrocarbons include phenol, benzene, cyclohexanone, cyclohexanol, cyclohexane, and cyclohexene.

Preferred end-products from the methods of the invention (both after purification of phenolic compounds and as according to the in situ method), are phenol and KA-oil (mixture of cyclohexanol and cyclohexanone).

The various embodiments described in connection with the depolymerization reaction, surprisingly, are equally applicable to the defunctionalization reaction of the invention.

The description refers to various embodiments and preferred embodiments. These various embodiment are not intended to be read merely in isolation. Rather, the skilled person will understand that generally one or more of these embodiments can be applied in combination.

The invention will hereinafter be illustrated with reference to the following non-limiting examples.

### General Procedures

### Lignin Depolymerization

P1000 mixed wheat straw/Sarkanda grass soda lignin (Greenvalue SA, Switzerland) (5.00 g) and demineralized water (50 mL) were placed in a 100 mL Parr Hastelloy reactor. If used, 1.00 g catalyst was added (e.g. 10% Pd on activated wood carbon, reduced, 50% wet paste, uniform precious metal distribution, BASF Escat 1931). The resulting suspension was adjusted to the desired pH by adding aqueous sodium hydroxide. Above pH 10 the lignin was completely dissolved. After the reactor was closed, stirring (500 rpm) was started and the reactor was heated to the desired temperature. After reaction, the reactor was rapidly cooled down to room temperature using a water bath. After opening the reactor, the resulting pH was measured. In order to re-dissolve any residual lignin, the pH was re-adjusted to the starting pH by adding aqueous sodium hydroxide. The reaction mixture was then filtered to remove, if present, the catalyst and/or char. Residues were dried in a vacuum oven at 40° C. for 18 h to determine catalyst loss or the amount of formed char.

To precipitate the residual lignin, the pH was adjusted to pH 3 by adding concentrated hydrochloric acid and the mixture was allowed to stand in the refrigerator for 18 h. The precipitated lignin was removed by centrifugation and dried in a vacuum oven at 40° C. for 18 h to determine the yield. The acidic water layer was extracted with chloroform (3×50 mL) to remove the low molecular weight compounds. The combined organic layers were dried (magnesium sulfate) and filtered. The solvent was removed by a rotary evaporator at 40° C. under reduced pressure. The extracted products were weighed and analyzed by GC-MS.

### Hydrothermal Defunctionalization of Phenolic Compounds

Reactions were carried out in 75 mL Parr MRS 5000 Hastelloy pressure reactors. In a typical procedure, demineralized water (40 mL), guaiacol (0.73 g, 6 mmol), catalyst (3 mole % metal) and magnetic stirring bars were introduced into the reactors. If desired, the pH was adjusted to the desired pH by the addition of a few drops of 4N NaOH. The reactors were closed and flushed 3 times with nitrogen (3.0). Stirring (500 rpm) was started and the reactors were heated to the desired temperature for 0 to 20 h. Next, the reaction mixtures were allowed to cool down to room temperature. In general, almost no overpressure was registered. After opening the reactors, diethyl ether (20 mL) was added and the pH was adjusted to pH 3 with 2N HCl. The organic phase was separated from the aqueous phase, the water layer was extracted 2× with 20 mL diethyl ether, and the combined organic layers were filtered to remove the catalyst. The water layer was filtered afterwards over the same filter to remove the remaining catalyst. The catalyst was dried (vacuum, 40° C., overnight), in order to determine the catalyst weight after reaction. The organic layer was dried over magnesium sulfate, filtered and transferred directly into 100 mL volumetric flasks. Samples were analyzed by GC-MS (TIC), and quantified by using hexadecane as an internal standard.

### Example 1

Depolymerization of P1000 soda lignin was performed as described under the general procedure. The results of the lignin depolymerization are shown in table 1.

TABLE 1
Results of the lignin depolymerization reactions.
Conditions: P1000 Lignin (5.00 g), demineralized water (50 mL), catalyst (1.00 g), 200-300° C., 0-24 h.

| | | | | | | | | | | Monomeric | | | | | |
|---|---|---|---|---|---|---|---|---|---|---|---|---|---|---|---|
| | | | | | | CHCl₃ | Residual | | Mass | compounds^{[a]} | | | | | |
| | | Time | Temp | pH | pH | solubles | Lignin | char | balance | 1 | 2 | 3 | 4 | 5 | 1-5 |
| Example | Catalyst | (h) | (° C.) | (start) | (after) | (wt. %) | (wt. %) | (wt. %) | (wt. %) | (%) | (%) | (%) | (%) | (%) | (%) |
| 1 | — | 4 | 250 | 10 | 6.7 | 8 | 54 | n.d. | 62 | 7 | 35 | 24 | 4 | 11 | 81 |
| 2 | Activated | 4 | 250 | 10 | 6.9 | 15 | 30 | 39 | 84 | 9 | 43 | 23 | 3 | 6 | 84 |
| | Carbon | | | | | | | | | | | | | | |
| 3 | 10% Pd/C | 4 | 250 | 10.1 | 6.7 | 18 | 37 | 18 | 73 | 8 | 53 | 8 | 7 | 4 | 80 |
| 4 | — | 4 | 250 | 11 | 7.8 | 20 | 67 | n.d. | 87 | 9 | 51 | 2 | 13 | 9 | 81 |
| 5 | 10% Pd/C | 4 | 250 | 11.2 | 7.4 | 18 | 46 | 15 | 79 | 10 | 47 | — | 14 | 8 | 79 |
| 6 | 10% Pd/C | 4 | 250 | 3.7 | 4.4 | 14 | 14 | 21 | 49 | 3 | 19 | 15 | — | 22 | 59 |
| 7 | 10% Pd/C | 0 | 250 | 10 | 7.5 | 10 | 72 | 9 | 91 | 4 | 27 | 30 | <1 | 16 | 77 |
| 8 | 10% Pd/C | 2 | 250 | 10 | 7.1 | 11 | 43 | 25 | 79 | 8 | 42 | 17 | 2 | 10 | 79 |
| 9 | 10% Pd/C | 24 | 250 | 10 | 6.9 | 6 | 21 | 35 | 63 | 11 | 31 | 3 | 7 | 0 | 48 |
| 10 | 10% Pd/C | 4 | 200 | 10 | 7.8 | 5 | 83 | 6 | 94 | 4 | 28 | 17 | 0 | 22 | 71 |
| 11 | 10% Pd/C | 4 | 300 | 11 | 6.9 | 5 | 39 | 15 | 59 | 17 | 16 | 3 | 3 | 0 | 39 |

^{[a]}Compounds were identified by GC-MS, and are presented as the % (TIC) of the total amount of detected monomeric compounds;
Compound numbers correspond to the following compounds: 1 = phenol, 2 = guaiacol, 3 = syringol, 4 = veratrole, 5 = acetosyringone.

### Example 2

Hydrothermal defunctionalization of phenolic compounds was performed as described under the general procedure. The results of the defunctionalization of phenolic compounds are shown in table 2.

TABLE 2
Results of the hydrothermal defunctionalization of phenolic compounds.
Conditions: Guaiacol (0.73 g, 5.88 mmol) or phenol (0.559 g, 5.88 mmol), demineralized water (40 mL), supported metal
catalyst (3 mole % metal relative to substrate), 200-300° C., 0-24 h.

| | | | | | | | Reaction product | | | | | | |
|---|---|---|---|---|---|---|---|---|---|---|---|---|---|
| | | | | | | | 1 | 2 | 3 | 4 | 5 | 6 | Mass |
| | | | Temp | Time | | Conversion | (mole | (mole | (mole | (mole | (mole | (mole | balance |
| Example | Substrate | Catalyst | (° C.) | (h) | pH | (mole %) | %) | %) | %) | %) | %) | %) | (mole %) |
| 1 | Guaiacol | 10% Pd/C | 250 | 20 | 5.4 | 18.6 | 4.4 | 0.8 | 0.0 | 0.0 | 0.2 | trace | 86.8 |
| 2 | Guaiacol | 10% Pd/C | 250 | 20 | 8 | 30.2 | 7.4 | 0.7 | 0.0 | 0.0 | 0.2 | trace | 78.1 |
| 3 | Guaiacol | 10% Pd/C | 250 | 20 | 9 | 35.8 | 9.4 | 0.9 | 0.0 | 0.0 | 0.3 | trace | 74.8 |
| 4 | Guaiacol | 10% Pd/C | 250 | 20 | 10 | 53.5 | 9.7 | 1.6 | 0.0 | 0.0 | 0.2 | trace | 58.0 |
| 5 | Guaiacol | 10% Pd/C | 250 | 20 | 10.5 | 99.5 | 27.8 | 3.0 | 14.0 | 6.7 | 8.3 | trace | 60.3 |
| 6 | Guaiacol | 10% Pd/C | 250 | 20 | 11 | 99.8 | 24.8 | 2.5 | 16.0 | 7.5 | 8.9 | trace | 59.9 |
| 7 | Guaiacol | 10% Pd/C | 250 | 0 | 11 | 8.0 | 2.5 | 0.2 | 0.0 | 0.0 | 0.0 | trace | 94.7 |
| 8 | Guaiacol | 10% Pd/C | 250 | 1 | 11 | 34.8 | 16.1 | 0.7 | 0.6 | 0.1 | 0.1 | trace | 82.8 |
| 9 | Guaiacol | 10% Pd/C | 250 | 4 | 11 | 71.6 | 34.6 | 1.8 | 3.8 | 0.5 | 0.8 | trace | 69.9 |
| 10 | Guaiacol | 10% Pd/C | 200 | 20 | 11 | 63.1 | 37.8 | 0.8 | 3.7 | 0.8 | 0.3 | trace | 80.2 |
| 11 | Guaiacol | 10% Pd/C | 225 | 20 | 11 | 96.9 | 38.1 | 1.5 | 13.6 | 5.3 | 2.5 | trace | 64.1 |
| 12 | Guaiacol | 10% Pd/C | 275 | 20 | 11 | 98.2 | 25.4 | 1.6 | 5 | 1.2 | 24.6 | trace | 59.6 |
| 13 | Guaiacol | 10% Pd/C | 225 | 4 | 11 | 47.4 | 49.6 | 1.7 | 1.7 | 0.0 | 0.2 | trace | 105.8 |
| 14 | Guaiacol | Act. Carbon | 250 | 20 | 11 | 69.9 | 0.1 | 0.0 | 0.0 | 0.0 | 0.0 | 0.0 | 30.2 |
| 15 | Phenol | 10% Pd/C | 250 | 20 | 11 | 3.7 | 96.3 | 0.0 | 0.0 | 0.0 | 0.0 | 0.0 | 96.3 |
| 16 | Guaiacol | 5% Pd/C | 250 | 20 | 11 | 98.6 | 10.5 | 0.0 | 24.1 | 13.6 | 21.5 | 0.0 | 71.1 |
| 17 | Guaiacol | 5% Pt/C | 250 | 20 | 11 | 99.2 | 39.8 | 0.2 | 0.0 | 0.0 | 0.0 | 1.9 | 42.7 |
| 18 | Guaiacol | 5% Ru/C | 250 | 20 | 11 | 97.7 | 1.4 | 0.0 | 0.0 | 1.3 | trace | 19.3 | 24.3 |
| 19 | Guaiacol | 5% Rh/C | 250 | 20 | 11 | 100.0 | 0.0 | 0.0 | 0.0 | 0.0 | 0.0 | 13.3 | 13.3 |

[a] Reaction products were identified and quantified GC-MS (TIC) using hexadecane as an internal standard.
Product yields are calculated in mole % relative to the amount of substrate;
Compound numbers correspond to the following compounds: 1 = phenol, 2 = anisole, 3 = cyclohexanone, 4 = cyclohexanol, 5 = cyclohexane, 6 = benzene.

What is claimed is:

1.-6. (canceled)

7. A process for the production of six-membered cyclic hydrocarbon compounds from lignin, the method comprising subjecting lignin to a depolymerization method according to any one of the claims 1 to 5, so as to obtain one or more phenolic compounds, and subjecting the phenolic compounds to a catalyzed hydrothermal defunctionalization reaction, said reaction being conducted in the presence of water at an alkaline pH>8 and a temperature of 200° C.-300° C., under the influence of a noble metal catalyst on a carbon support.

8. A process according to claim 7, wherein the defunctionalization reaction is conducted at a pH of 9-12, preferably at a pH of 10-11.

9. A process according to claim 7 or 8, wherein the defunctionalization reaction is conducted for a period of 1 to 6 hours, preferably 2 to 4 hours.

10. A process according to any one of the claims 7 to 9, wherein the defunctionalization reaction is conducted at a temperature of from 225° C. to 275° C., preferably at 250° C.

11. A process according to any one of the claims 7 to 10, wherein the noble metal catalyst used in the defunctionalization reaction is selected from the group consisting of palladium, platinum, ruthenium, and rhodium.

12. A process according to any one of the claims 7 to 11 for the production, from lignin, of one or more six-membered cyclic hydrocarbons selected from the group consisting of phenol, benzene, cyclohexanone, cyclohexanol, cyclohexane, and cyclohexene, the process comprising subjecting one or more phenolic compounds selected from 2-methoxy phenol, 2,5-dimethoxy phenol, and mixtures thereof to the catalyzed hydrothermal defunctionalization reaction, so as to obtain said one or more hydrocarbons.

13. A process according to claim 12, for the production of phenol from one or more phenolic compounds are selected from the group consisting of 2-methoxy phenol, 2,5-dimethoxy phenol, and mixtures thereof.

14. A process for the defunctionalization of one or more phenolic compounds selected from the group consisting of phenol, C₁₋₆ alkyl alkoxy phenols, and acetosyringone (4-hydroxy-3,5-dimethoxyacetophenone), comprising subjecting the one or more phenolic compounds to a catalyzed hydrothermal defunctionalization reaction, said reaction being conducted in the presence of water at an alkaline pH>8 and a temperature of 200° C.-300° C., under the influence of a noble metal catalyst on a carbon support.

15. A process according to claim 14, wherein the alkyl and the alkoxy groups in the C₁₋₆ alkyl alkoxy phenol each independently are C₁₋₃ alkyl or alkoxy, preferably methyl or methoxy.
